(19) United States
(12) Patent Application Publication
(10) Pub. No.: US 2025/0228965 A1
(43) Pub. Date: Jul. 17, 2025

(54) NANOPARTICLES COMPRISING AT LEAST ONE METAL SALT AND AT LEAST ONE PEPTIDE

(71) Applicants: Centre National de la Recherche Scientifique, Paris (FR); Université Paris Nord, Villetaneuse (FR); Institut National de la Santé et de la Recherche Médicale (INSERM), Paris (FR); Université d'Angers, Angers (FR)

(72) Inventors: Célia ARIB, Houilles (FR); Jolanda SPADAVECCHIA, Melun (FR); Audrey GRIVEAU, Angers (FR); Joël EYER, Blaison-Gohier (FR)

(21) Appl. No.: 18/692,585

(22) PCT Filed: Sep. 15, 2022
(86) PCT No.: PCT/EP2022/075636

§ 371 Date: Mar. 15, 2024

(30) Foreign Application Priority Data
Sep. 15, 2021 (FR) FR2109696

(51) Int. Cl. A61K 47/69; A61K 33/242; A61K 47/64

(52) U.S. Cl. CPC A61K 47/6935; A61K 33/242; A61K 47/64

(57) ABSTRACT

The present invention relates to a nanoparticle comprising:
an association of at least one metal salt and at least one peptide; and
at least one biocompatible polymer.

### INVENTION FIELD The present invention relates to a nanoparticle and its use as a drug or diagnostic agent. The present invention also relates to a process for preparing a nanoparticle.

### TECHNICAL BACKGROUND

Hybrid nanoparticles combining a metallic part with a biocompatible polymer and a therapeutic molecule are promising nanomedicines and theranostic agents, particularly for cancer treatment. These nanoparticles can both transport therapeutic molecules to target cells and, thanks to their metallic part, serve as photo-thermo-therapeutic agents and/or in vivo imaging markers.

Thus, Emami et al (2019) Mol. Pharmaceutics 16: 1184-1199 describe the use of hybrid nanoparticles combining a gold nanoparticle, a biocompatible polymer, polyethylene glycol (PEG), a therapeutic molecule, doxorubicin, and an anti-PD-L1 antibody, enabling the targeting of colorectal cancer cells that over-express PD-L1, to destroy cells of the CT-26 colorectal cancer cell line. The authors show that these nanoparticles facilitate doxorubicin entry into CT-26 cells, with 66% of cells undergoing apoptosis, and that the combination of doxorubicin treatment and near-infrared irradiation significantly and synergistically abolishes in vitro proliferation of CT-26 cells by enhancing apoptosis and cell cycle arrest. However, the photo-thermo-therapeutic efficacy of these nanoparticles is not optimal. Indeed, the same nanoparticles without doxorubicin only induce cell death in around 20% of cells after near-infrared irradiation. In addition, the combination of doxorubicin and near-infrared irradiation only reduces the survival rate of CT-26 cells from around 20% to around 10%, compared with the same treatment in the absence of irradiation.

As a result, the full therapeutic potential of this type of hybrid nanoparticle has yet to be realized, and it would therefore be useful to have hybrid nanoparticles capable of delivering therapeutic molecules with enhanced photo-thermo-therapeutic properties.

### SUMMARY OF THE INVENTION

The present invention arises from the unexpected discovery by the inventors that hybrid nanoparticles obtained by chelating chlorauric acid with a cell-penetrating peptide, in particular one of the NFL, TAT or VIM peptides, functionalized with a biotin and with dicarboxylic acid-terminated polyethylene glycol (NFL-BIOTINE-PEG-AuNPs, TAT-BIOTINE-PEG-AuNPs, VIM-BIOTINE-PEG-AuNPs) exhibit high stability and biocompatibility and are particularly suitable for cancer treatment.

Advantageously, the process for obtaining nanoparticles according to the invention, in particular NFL-BIOTINE-PEG-AuNPs, TAT-BIOTINE-PEG-AuNPs, and VIM-BIOTINE-PEG-AuNPs nanoparticles, is a simple process comprising few steps.

The inventors have demonstrated that NFL-BIOTINE-PEG-AuNPs hybrid nanoparticles at concentrations of 500 μmol/L and 1000 μmol/L significantly reduce the cell viability of MiaPaCa-2 cells (a human pancreatic cancer cell line) and F98 cells (a rat glioblastoma cell line). NFL-BIOTINE-PEG-AuNPs hybrid nanoparticles at 100 μmol/L also significantly decrease cell viability of MiaPaCa-2 cells and F98 cells irradiated after nanoparticle administration.

The inventors have also demonstrated that TAT-BIOTINE-PEG-AuNPs and VIM-BIOTINE-PEG-AuNPs hybrid nanoparticles significantly reduce cell viability of F98 cells (a rat glioblastoma cell line).

Thus, the present invention relates to a nanoparticle comprising:
an association of at least one metal salt and at least one peptide; and
at least one biocompatible polymer.

The present invention also relates to a nanoparticle as defined above for use as a drug and/or diagnostic agent.

The present invention also relates to a pharmaceutical composition comprising at least one nanoparticle as defined above as active ingredient, optionally in association with a pharmaceutically acceptable vehicle.

The present invention also relates to a diagnostic composition comprising at least one nanoparticle as defined above.

The present invention also relates to a medical device comprising at least one nanoparticle as defined above.

The present invention also relates to a process for preparing a nanoparticle comprising:
an association of at least one metal salt and at least one peptide; and
at least one biocompatible polymer,

comprising:
a step wherein the metal salt is chelated with the peptide;
a step of mixing with the biocompatible polymer; and
a step of reducing the mixture with a reducing agent.

### DETAILED DESCRIPTION OF THE INVENTION

As used here, the term “comprising” is synonymous with “including”, “containing” or “encompassing”, i.e. when an object “comprises” one or more elements, elements other than those mentioned can also be included in the object. Conversely, “consisting of” means “constituted by”, i.e. when an object “consists of” one or more elements, the object cannot include elements other than those mentioned.

As used herein, “biocompatible” in the sense of the invention means the ability of a material to be compatible with a biological medium, in particular a tissue or fluid of a living organism. Thus, a biocompatible material in the sense of the invention can be used in a pharmaceutical composition, in a diagnostic composition, in a medical device, for example for diagnostic or therapeutic purposes.

As used herein, “biopolymer” or “natural polymer” in the sense of the invention is a polymer that can be produced by a living organism, such as an animal, plant or fungus.

### Metal Salt

For the purposes of this invention, metal salts are defined as metal oxides and salts produced by the action of an acid on a metal. Preferably, the metal salt according to the invention is derived from the action of an acid on a metal.

Preferably also, the metal salt according to the invention is selected from the group consisting of a transition metal salt, a lanthanide salt, an alkaline earth metal salt and mixtures thereof.

More preferably, the metal salt according to the invention is selected from the group consisting of a salt of gold (Au), selenium (Se), gadolinium (Gd), cobalt (Co), europium (Eu), terbium (Tb), cerium (Ce), manganese (Mn), iron (Fe), zinc (Zn) and copper (Cu).

Preferably also, the metal salt according to the invention is an inorganic acid metal salt. Examples of inorganic acid metal salts according to the invention include sulfates, chlorides, nitrates, phosphates and carbonates.

More preferably, the metal salt according to the invention is chloroauric acid HAuCl₄.

According to a particular embodiment of the invention, the metal salt according to the invention is not an iron salt or an iron oxide.

### Peptide

Preferably, the peptide according to the invention is an isolated amino acid sequence comprising no more than 100 amino acids. Also preferably, the peptide according to the invention comprises between 2 and 100 amino acids, for example between 2 and 90, between 2 and 80, between 2 and 70, between 2 and 60, between 2 and 50 and between 2 and 40 amino acids. Preferably, the peptide according to the invention comprises at least 2 amino acids, for example at least 3, at least 4, at least 5, at least 6, at least 7, at least 8, at least 9 and at least 10 amino acids. More preferably, the peptide according to the invention comprises between 2 and 40 amino acids, for example, between 10 and 40 amino acids or between 10 and 30 amino acids.

The peptide according to the invention can be selected from any biocompatible peptide well known to the person skilled in the art. Preferably, the peptide according to the invention is selected from the group consisting of a chimeric peptide, a synthetic peptide and a peptide derived from a protein.

Preferably, the peptide according to the invention is a peptide capable of penetrating cells, in particular the plasma membrane of cells. More preferably, the peptide according to the invention is a cell penetrating peptide (CPP).

Preferably also, the peptide according to the invention is a peptide capable of crossing the blood-brain barrier.

Preferably, the peptide according to the invention is a cell penetrating peptide selected from the group consisting of a polycationic peptide with a sequence rich in positively charged amino acid residues such as lysine or arginine, an amphiphilic peptide with a sequence alternating electrically charged polar residues and hydrophobic apolar residues, and a hydrophobic peptide containing only weakly charged apolar or hydrophobic residues.

Preferably, the peptide according to the invention is selected from the group consisting of the NFL peptide having the following amino acid sequence: YSSYSAPVSSSLSVRRSYSSSSGS (SEQ ID NO: 1), the TAT peptide having the following amino acid sequence: GRKKRRQRRRPPQ (SEQ ID NO: 2), the VIM peptide having the following amino acid sequence: GGAYVTRSSAVRLRSSVPGVRLLQ (SEQ ID NO: 3), the peptide transportane having the following amino acid sequence: GWTLNSAGYLLGKINLKALAALAKKIL (SEQ ID NO: 4), the penetratin peptide having the following amino acid sequence: RQIKIWFQNRRMKWKK (SEQ ID NO: 5), the MAP peptide having the following amino acid sequence: KLALKLALKALKAALKLA (SEQ ID NO: 6), the Pep-1 peptide following amino acid sequence: KETWWETWWTEWSQPKKKRKV (SEQ ID NO: 7), the Pept 1 peptide having the following amino acid sequence: PLILLRLLRGQF (SEQ ID NO: 8), the Pept 2 peptide having the following amino acid sequence: PLIYLRLLRGQF (SEQ ID NO: 9), the IVV-14 peptide with the following amino acid sequence: KLWMRWYSPTTRRYG (SEQ ID NO: 10), the Ig(v) peptide having the following amino acid sequence: MGLGLHLLVLAAALQGAKKKRKV (SEQ ID NO: 11), the pVEC peptide having the following amino acid sequence: LLIILRRRIRKQAHAHSK (SEQ ID NO: 12), the ppTG20 peptide having the following amino acid sequence: GLFRALLRLLRSLWRLLLRA (SEQ ID NO: 13), the APP peptide having the following amino acid sequence: APP (GLARALTRLLRQLTRQLTRA) (SEQ ID NO: 14), the peptide having the following amino acid sequence RLWMRWYSPRTRAYGC (SEQ ID NO: 15); of the peptide having the following amino acid sequence: WRWYCR (SEQ ID NO: 16); the PepFect 14 peptide having the following amino acid sequence: stearyl-AGYLLGKLLOOLAAAALOOLL-NH2 (SEQ ID NO: 17); of the peptide having the following amino acid sequence: GWTLNSAGYLLGKINLKALAALAKKIL (SEQ ID NO: 18), the HRSV peptide having the following amino acid sequence: RRIPNRRPRR (SEQ ID NO: 19) or a derivative thereof.

The peptide according to the invention can also be a cyclic peptide such as, for example, the cyclic RGD peptide.

More preferably, the peptide according to the invention is selected from the group consisting of the NFL peptide having the following amino acid sequence: YSSYSAPVSSSLSVRRSYSSSSGS (SEQ ID NO: 1), the TAT peptide having the following amino acid sequence: GRKKRRQRRRPPQ (SEQ ID NO: 2), the VIM peptide with amino the following acid sequence: GGAYVTRSSAVRLRSSVPGVRLLQ (SEQ ID NO: 3) or a derivative thereof.

According to one embodiment of the invention, the peptide according to the invention is not a peptide targeting the EGFR receptor, in particular a-lysine-azidoacetic acid peptide, or a peptide targeting bone.

As used herein, peptide derivative in the sense of the invention refer to variants and fragments of the peptide to which they refer. Preferably, the derivatives, in particular the variants or fragments, of the peptide according to the invention are biologically active.

Biologically active derivatives of the peptide according to the invention are variants and fragments that retain the biological activity and specificity of the parent peptide.

The variant of the peptide according to the invention can be chosen from any variant well known to the person skilled in the art. An example of a variant according to the invention is an allelic variant of the peptide, and a peptidomimetic variant of the peptide.

An allelic variant of the peptide according to the invention has the same amino acid sequence as the peptide according to the invention, in particular as SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, or SEQ ID NO: 19 except that one or more amino acids have been replaced by other amino acids or deleted, with the final peptide retaining the specificity of the parent peptide.

Preferably, the peptide according to the invention has at least 70%, at least 75%, at least 80%, at least 85%, at least 90%, at least 95%, at least 98% identity to the parent peptide, in particular to SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18 or SEQ ID NO: 19.

Preferably also, the variant according to the invention has the same number of amino acids as the peptide from which it is derived.

The percentage of identity between two peptide sequences is well known to the person skilled in the art. By way of example, the percentage of identity between two peptide sequences can be determined by performing a sequence alignment along the entire length of similar sequences and determining the number of positions at which the amino acids are identical in each sequence, and dividing this number by the total number of amino acids in the longer sequence.

The peptidomimetic variant according to the invention is preferably an organic molecule which mimics certain properties of the parent peptide. Preferred peptidomimetic variants according to the invention are obtained by structural modification of the peptides according to the invention, for example using non-natural amino acids such as a D-amino acid instead of an L-amino acid, conformational constraints, isosteric replacement, cyclization or other modifications.

Examples of other modifications include the following: replacement of one or more amide bonds by a non-amide bond; replacement of one or more amino acid side chains by a different chemical moiety; protection of one or more N-terminal, C-terminal ends or side chain by a protecting group; introduction of a double bond or ring into the main chain. Advantageously, the modifications have the effect of increasing the rigidity, binding affinity, resistance to enzymatic degradation, bioavailability and, more generally, of improving the pharmacokinetic properties of the peptide according to the invention.

Thus, taking into account the peptide sequences of the peptide according to the invention, the person skilled in the art is able to design and produce peptidomimetic variants with similar or superior biological characteristics to the peptides according to the invention, in particular to the cell penetrating peptides, more particularly to the NFL, TAT and VIM peptides according to the invention.

The NFL peptide, also referred to as NFL-TBS40-63 hereafter, is a 24-amino acid polypeptide with the following sequence: YSSYSAPVSSSLSVRRSYSSSSGS (SEQ ID NO: 1). This peptide is well known to the person skilled in the art and corresponds to the second tubulin-binding site of the light neurofilament subunit (amino acids 40 to 63 of the NFL protein TBS site).

In one embodiment of the invention, the peptide according to the invention is a derivative of the NFL peptide, in particular a variant or fragment thereof. Preferably, fragments and variants of the NFL peptide are biologically active.

Preferably, the NFL peptide fragment according to the invention comprises at least 12 successive amino acids of the parent NFL peptide, preferably at least 16, more preferably at least 18 amino acids.

Preferably, the variant according to the invention comprises at least 70%, at least 75%, at least 80%, at least 85%, at least 90%, at least 95%, at least 98% identity to the NLF peptide. An example of an allelic variant of the NFL peptide is the TBS motif of the light subunit of the quail neurofilament, which retains 20 out of 24 amino acids of the NFL-TBS40-63 peptide.

The TAT peptide, hereinafter also referred to as TAT48-60, is a 13-amino acid peptide with the following sequence GRKKRRQRRRPPQ (SEQ ID NO: 2). This peptide, well known to the person skilled in the art, corresponds to residues 48 to 60 of the TAT protein involved in human immunodeficiency virus type 1 (HIV-1) replication.

In one embodiment of the invention, the peptide according to the invention is a derivative of the TAT peptide, in particular a variant or fragment thereof. Preferably, the TAT peptide derivative according to the invention is biologically active.

Preferably, the TAT peptide fragment according to the invention comprises at least 5 successive amino acids of the TAT parent peptide, preferably at least 6, at least 7, at least 8 amino acids.

Preferably, the variant according to the invention comprises at least 70%, at least 75%, at least 80%, at least 85%, at least 90%, at least 95%, at least 98% identity to the TAT peptide.

The VIM peptide is a 24 amino acid peptide with the following sequence: GGAYVTRSSAVRLRSSVPGVRLLQ (SEQ ID NO: 3).

In one embodiment of the invention, the peptide according to the invention is a derivative of the VIM peptide, in particular a variant or fragment thereof. Preferably, the VIM peptide derivative according to the invention is biologically active.

Preferably, the VIM peptide fragment according to the invention comprises at least 12 successive amino acids of the parent VIM peptide, preferably at least 14, at least 16 and more preferably at least 18 amino acids.

Preferably, the VIM peptide variant according to the invention comprises at least 70%, at least 75%, at least 80%, at least 85%, at least 90%, at least 95%, at least 98% identity to the VIM peptide.

The penetratin peptide is a 16-residue long fragment with the following amino acid sequence: RQIKIWFQNRRMKWKK (SEQ ID NO: 5). This peptide, well known to the person skilled in the art, is derived from the homeodomain of the Drosophila Antennapedia transcription factor.

In one embodiment of the invention, the peptide according to the invention is a derivative of the penetratin peptide, in particular a variant or fragment thereof. Preferably, the penetratin peptide derivative according to the invention is biologically active.

Preferably, the penetratin peptide fragment according to the invention comprises at least 5 successive amino acids of the penetratin parent peptide, preferably at least 6, at least 7, at least 8 amino acids.

Preferably, the variant according to the invention comprises at least 70%, at least 75%, at least 80%, at least 85%, at least 90%, at least 95%, at least 98% identity with respect to the penetratin peptide.

The peptide according to the invention can be prepared by any technique well known to the person skilled in the art, in particular by chemical synthesis.

According to one embodiment, the peptide as defined above is coupled with at least one coupling agent. As understood herein, the terms “coupling agent” and “conjugating agent” are interchangeable. As understood herein, the terms “coupled”, “conjugated”, “functionalized” and “modified” may be used interchangeably.

The coupling agent according to the invention can be chosen from any coupling agent well known to the person skilled in the art.

Preferably, the coupling agent according to the invention is non-peptidic.

The coupling agent can be bifunctional, preferably heterobifunctional, such as N-γ-maleimidobutyryl-oxysuccinimide ester (GMBS) and sulfo-GMBS derivative, m-maleimidobenzoyl-n-hydroxysuccinimide ester (MBS) and sulfo-MBS derivative, succinimidyl 4-(N-maleimidomethyl)cyclohexane-1-carboxylate (SMCC), sulfo-succinimidyl4-(N-maleimidomethyl) cyclohexane-1-carboxylate (sulfo-SMCC), carbodiimide, bisdiazonium-benzidine (BDB) or glutaraldehyde, biotin, sulfo-NHS-bitoin or a fluorophore, also known as a fluorochrome, such as hydroxycoumarin, aminocoumarin, methoxycoumarin, Cascade Blue, Pacific Blue, Pacific Orange, Lucifer Yellow, NBD, RPhycoerythrin, PE-Cy5 conjugates, PE-Cy7 conjugates, Red 613, PerCP, TruRed, FluorX, fluorescein, BODIPY-FL, TRITC, X-Rhodamine, Lissamine Rhodamine B, Texas Red, Allophycocyanine, APC-Cy7 conjugates, 5-carboxytetramethylrhodamine, 5.6-carboxytetramethylrhodamine, 6-carboxytetramethylrhodamine, 5-carboxyfluorescein, 5,6-carboxyfluorescein, 6-carboxyfluorescein, and dimethylaminonaphthylpyridinium.

The use of coupling agents is described in particular in the chapter “Production of Antisera Using Peptide Conjugates” in the reference work “The Protein Protocols Handbook” (2002), which is incorporated here by reference.

When GMBS, MBS, SMCC or sulfo-SMCC are used, they are preferably attached to a cysteine (C), which if not present in the peptide sequence, can be added, particularly at its N-terminal or C-terminal end.

The coupling agent according to the invention can also be chosen from any functional group well known to the person skilled in the art which can bind to a peptide. Examples of functional groups according to the invention include fluorine, a phosphate group, an acetyl group, an alkyl group, a glutamic acid residue, a carboxyl group, a glycine residue, a glycosyl group, a hydroxyl group, an amide group and a sulfate group.

Preferably, the coupling agent according to the invention is selected from the group consisting of biotin and fluorescein.

In one embodiment, the peptide according to the invention is functionalized by at least one biotin, for example by one biotin, by two biotins or by three biotins.

In another embodiment, the peptide according to the invention is functionalized with at least one fluorescein, for example one fluorescein, two fluoresceins or three fluoresceins.

The peptide according to the invention can be modified by any conventional peptide coupling process well known to the person skilled in the art, for example chemically or enzymatically. Examples of processes for modifying the peptide according to the invention include acetylation, alkylation, biotinylation, glutamylation, glycylation, glycosylation, hydroxylation, phosphorylation, sulfation, amidation and PEGylation.

Preferably, the coupling agent according to the invention, in particular fluorescein or biotin, is covalently bound to the peptide according to the invention.

Functionalization of the peptide according to the invention with at least one coupling agent, in particular fluorescein or biotin, can be carried out directly on a primary amine, a secondary amine or both.

In one embodiment of the invention, at least one coupling agent, in particular fluorescein or biotin, is bound to the N-terminus or C-terminus of the peptide according to the invention.

In one embodiment of the invention, functionalization of the peptide with at least one coupling agent, in particular fluorescein or biotin, is carried out with an intermediate well known to the person skilled in the art on the primary or secondary carboxylic functions or both. An example of a bonding intermediate is a carbodiimide such as 1-ethyl-3-(3-dimethylaminopropyl) carbodiimide.

### Biocompatible Polymer

The biocompatible polymer according to the invention can be a natural or synthetic polymer and can be selected from any biocompatible polymer well known to the person skilled in the art.

Preferably, the biocompatible polymer according to the invention is selected from the group consisting of synthetic biocompatible polymers, biopolymers and heat-sensitive biocompatible polymers.

More preferably, the biocompatible polymer according to the invention is selected from the group consisting of polyethylene glycol (PEG), polyethylene glycol diacid (PEG-diacid), polyethylene glycol diamine (PEG-diamine), collagen, alginate, elastin, hyaluronic acid, cellulose, gelatin, polylactic acid, maltodextrin, glucose polymer, lactose polymer, chitosan, dextran, thermosensitive polymer, poly(N-isopropylacrylamide), poly[2-(dimethylamino)ethyl methyl methacrylate] (pDMAEMA), hydroxypropyl cellulose, poly(vinylcaprolactam) and polyvinyl methyl ether) or a combination thereof.

More preferably, the biocompatible polymer is dicarboxylic polyethylene glycol.

### Nanoparticle

Preferably, the nanoparticle according to the invention has a diameter between 5 nm and 500 nm, more preferably between 5 nm and 200 nm for example between 5 nm and 150 nm, between 5 nm and 100 nm, between 5 nm and 90 nm. More preferably, the nanoparticle according to the invention has a diameter between 20 nm and 200 nm, for example between 20 nm and 150 nm, or between 20 nm and 100 nm.

Preferably, the peptide according to the invention is inside the nanoparticle according to the invention. Thus, advantageously, the peptide adopts a steric configuration enabling the therapeutic activity of the peptide to be preserved and/or enhanced without impacting the stability of the nanoparticle.

Preferably, the metal salt is chelated by the peptide according to the invention.

Preferably, the metal salt and peptide are encapsulated by the polymer.

Preferably, the nanoparticle according to the invention is prepared according to the process comprising the following steps:
a step wherein the metal salt is chelated with the peptide;
a step of mixing with the biocompatible polymer; and
a step of reducing the mixture with a reducing agent.

Preferably, the step in which the metal salt is chelated with the peptide comprises dissolving the metal salt, preferably in an aqueous solution, and adding the peptide to the metal salt solution.

Advantageously, the biocompatible polymer is used as a stabilizing agent.

Preferably, the nanoparticle preparation process according to the invention is carried out in the absence of any surfactant other than the biocompatible polymer, and in the absence of any chemical binder.

Preferably, the reducing agent according to the invention can be any reducing agent well known to the person skilled in the art. Examples of reducing agents include LiAlH₄, NaBH₄, NaHg, B₂H₆, SO₂. Preferably, the reducing agent according to the invention is NaBH₄.

The nanoparticle preparation process according to the invention can also include a step of centrifuging the nanoparticle solution, preferably after the reduction step, in order to remove excess of biocompatible polymer.

Advantageously, the nanoparticle according to the invention is stable. Preferably, the nanoparticle according to the invention is stable and its biological activity, in particular properties of targeting and cellular internalization of the peptide, is maintained for at least 2 months, 4 months, 6 months, 8 months, 10 months, 12 months, 14 months, 16 months, 18 months, 20 months, 22 months, or 24 months.

### Use

Nanoparticles according to the invention are particularly suitable for use as a drug and/or diagnostic agent in an individual.

Nanoparticles according to the invention are particularly suitable for use as theranostic agent.

Nanoparticles according to the invention are also particularly suitable for use in a medical device intended for use in an individual.

Thus, one embodiment of the present invention also relates to a pharmaceutical composition comprising at least one nanoparticle according to the invention as active ingredient, optionally in association with a pharmaceutically acceptable carrier or excipient.

The present invention also relates to the use of at least one nanoparticle according to the invention for the manufacture of a medicament, in particular a medicament for the prevention or treatment of cancer, especially for the prevention or treatment of glioblastoma or pancreatic cancer.

The present invention also relates to the use of at least one nanoparticle according to the invention for the targeting, detection and destruction of cancer cells.

The present invention also relates to a method of treating cancer, in particular glioblastoma or pancreatic cancer, in an individual comprising administering to the individual an effective amount of at least one nanoparticle or pharmaceutical composition or drug according to the invention.

The present invention also relates to a diagnostic composition comprising at least one nanoparticle according to the invention as active ingredient, optionally in association with a pharmaceutically acceptable carrier or excipient.

The medical device according to the invention preferably comprises at least one nanoparticle according to the invention. Preferably, the medical device according to the invention is used for the diagnosis, prevention or treatment of cancer, in particular glioblastoma or pancreatic cancer. The medical device according to the invention may be chosen from any medical device well known to the person skilled in the art. Preferably, the medical device according to the invention is selected from the group consisting of a medical imaging device or an implantable device such as an implant, stent or catheter. By implantable device, we mean any apparatus intended to be introduced wholly or partially, by medical intervention, into the human or animal body.

According to one embodiment of the invention the nanoparticle, pharmaceutical composition, drug or medical device according to the invention is used for the prevention or treatment of cancer, in particular glioblastoma or pancreatic cancer.

According to one embodiment of the invention, the nanoparticle, diagnostic composition or medical device according to the invention is used for detecting cancer cells or tumors, for diagnosing cancer, for monitoring the progress of cancer and/or for monitoring therapeutic treatment of cancer.

According to one embodiment of the present invention, the nanoparticle, pharmaceutical composition, diagnostic composition, drug, or medical device is used as a contrast agent for diagnostic imaging such as radiography, magnetic resonance imaging (MRI), Raman spectroscopy, optical imaging, optical coherence tomography, X-ray, computed tomography, positron emission tomography or combinations thereof.

One embodiment of the present invention relates to monitoring the administration of the nanoparticle, its biodistribution and bioavailability, of the pharmaceutical composition, drug, or diagnostic composition according to the invention to an individual by a diagnostic device and diagnosing the individual.

Preferably, the nanoparticles, the drug, the pharmaceutical or diagnostic composition, the medical device according to the invention are used in at least one of the following situations:
diagnose a cancer, particularly glioblastoma or pancreatic cancer;
determine the status or evolution of a cancer, particularly glioblastoma or pancreatic cancer,
treat a cancer, particularly glioblastoma or pancreatic cancer,
monitoring therapeutic treatment of a cancer, particularly glioblastoma or pancreatic cancer.

### Cancer

The cancer prevented or treated according to the invention can be any cancer known to the person skilled in the art.

The cancer prevented or treated according to the invention may be stage 1, stage 2, stage 3 or stage 4.

Preferably, the cancer prevented or treated according to the invention is selected from the group consisting of cancers of the central nervous system, the endocrine system and the digestive system.

Preferably, the cancer prevented or treated according to the invention is selected from the group consisting of thyroid cancer, parathyroid cancer, thymus cancer, ovarian cancer, testicular cancer, pancreatic islet cancer, gallbladder cancer, stomach cancer, esophageal cancer, colon cancer, glioblastoma, liver cancer, pancreatic cancer, rectal cancer, intestinal cancer, small bowel cancer. More preferably, the cancer prevented or treated according to the invention is selected from the group consisting of glioblastoma and pancreatic cancer.

According to one embodiment of the invention, the nanoparticle, pharmaceutical composition, drug, diagnostic composition or medical device according to the invention is particularly suitable for use in photothermal therapy, in particular, for the treatment of cancer by photothermia.

Photothermy is a method of cancer treatment well known to the person skilled in the art. Preferably, the nanoparticle according to the invention, the pharmaceutical composition or the drug according to the invention is injected into the bloodstream of the individual, preferably by intravenous injection. The nanoparticle then penetrates the tumor or cancer cells to be treated, which are exposed to infra-red radiation to destroy them.

### Pharmaceutical Composition

As used herein, “pharmaceutically acceptable carrier, vehicle or excipient” refers to any material suitable for use in a pharmaceutical composition.

Preferably, the pharmaceutically acceptable vehicle or excipient according to the invention is suitable for use with nanoparticles according to the invention, preferably in liquid form.

Preferably, the pharmaceutically acceptable vehicle or excipient according to the invention is suitable for oral, parenteral, intradermal, intravenous, arterial, intramuscular, nasal, rectal or subcutaneous administration.

Preferably, the pharmaceutically acceptable vehicle or excipient according to the invention includes, but is not limited to, any standard vehicle or excipient well known to the person skilled in the art, such as water, glycerine, alcohol, oil emulsion, water emulsion, physiological saline, buffer solution, preservative, stabilizer, etc.

### Individual

Preferably, the individual according to the invention is an animal, preferably a mammal, such as a human, a canine, in particular a dog, a feline, in particular a cat, an equine, a bovine, a porcine, a caprine, in particular a goat or a sheep, a camelid, a rodent in particular a mouse or a rat. More preferably, the individual according to the invention is a human.

Preferably, the individual according to the invention has a cancer, in particular a cancer of the central nervous system, a cancer of the endocrine system and a cancer of the digestive system.

More preferably, the individual according to the invention suffers from glioblastoma or pancreatic cancer.

Preferably also, the individual according to the invention is suspected of having cancer, in particular glioblastoma or pancreatic cancer.

### Administration

Preferably, the nanoparticle according to the invention, the pharmaceutical composition, the diagnostic composition, the drug, or the medical device according to the invention is administered in a prophylactically or therapeutically effective amount to detect, prevent or treat a cancer as defined above.

Preferably, the nanoparticle according to the invention, pharmaceutical composition, drug, diagnostic composition or medical device according to the invention can be administered orally, parenterally, intradermally, intravenously, arterially, intramuscularly, nasally, rectally or subcutaneously.

The nanoparticle according to the invention, pharmaceutical composition, drug, diagnostic composition or medical device may be formulated as an injectable suspension, gel, oil, suppository, capsule, etc. . . .

The nanoparticle according to the invention, pharmaceutical composition, drug, diagnostic composition or medical device can be administered to the individual in a dose between 10 μmol/L and 2000 μmol/L, preferably between 50 μmol/L and 1500 μmol/L, more preferably between 100 μmol/L and 1000 μmol/L, for example between 200 μmol/L and 1000 μmol/L, between 300 μmol/L and 1000 μmol/L between 400 μmol/L and 1000 μmol/L, between 500 μmol/L and 1000 μmol/L.

The person skilled in the art is able to adjust the dose of nanoparticle or pharmaceutical composition, diagnostic composition or drug according to the weight of the individual to be treated.

### Additional Compound

The nanoparticle according to the invention, pharmaceutical composition, drug, diagnostic composition or medical device can be administered with an additional compound for the prevention or treatment of cancer.

The nanoparticle according to the invention can be used as a carrier of an additional compound for the prevention or treatment of cancer. In particular, the nanoparticle according to the invention can be used to deliver an additional compound for the prevention or treatment of cancer intracellularly. Advantageously, the nanoparticle according to the invention can be used to transport a therapeutic molecule to target cells, in particular to the cancer cells of the tumor to be treated.

According to one embodiment, the invention relates to a product comprising:
at least one nanoparticle as defined above;
at least one additional compound for the prevention or treatment of cancer, in particular glioblastoma and pancreatic cancer as a combined preparation for use, notably simultaneously, separately or sequentially, for the prevention or treatment of cancer, in particular glioblastoma or pancreatic cancer.

As understood here, “combined” or “in combination” means that the nanoparticle according to the invention is administered at the same time as another compound, either together, i.e. at the same administration site, or separately or at different times, provided that the period of time during which the nanoparticle according to the invention exerts its effects in the individual and the period of time during which the additional compound produces its pharmacological effects in the individual at least partially overlap.

The additional compound for the prevention or treatment of cancer can be any compound well known to the person skilled in the art.

Preferably, the additional compound is selected from the group consisting of cyclophosphamide, docetaxel, doxorubicin, epirubicin, fluorouracil, methotrexate, paclitaxel, everolimus, alectinib, melphalan, brigatinib, nilutamide, cyproterone acetate, anastrozole, exemestane, lomustine, bosutinib, encorafenib, cabozatinib, vandenatib, bicalutamide, etopositde, chlorambucil, cobimetinib, enzalutamide, idarubicie, capecitabine, tamoxifen, dexamethasone, bisulfan, lenvatinib, gemcitabine, 5-FU, irinotecan, temozolomide, oxaliplatin, nab-paclitaxel and combinations thereof.

The invention will be further explained in a non-limiting manner with the aid of the following Figures and Examples.

### DESCRIPTION OF FIGURES

FIG. 1 shows the UV-visible spectrum of NFL-BIOTINE-PEG-AuNPs nanoparticle synthesis with wavelength (nm) versus absorbance (a.u).

FIG. 2 shows the UV-visible spectrum of NFL-BIOTINE-PEG-AuNPs nanoparticles at T0, and at T0+11 months (Test 1 and Test 2).

FIG. 3 shows the temperature rise (in ° C.) of water (H₂O), of a solution of the NFL peptide functionalized with a biotin (NFL-BIOT), of a solution of gold salt complex with biotin-functionalized peptide (AuNPs) and of a solution of NFL-BIOTIN-PEG-AuNPs nanoparticles as a function of time (in min).

FIG. 4 shows the mitochondrial activity of MiaPaCa-2 cells, colchicine-treated MiaPaCa-2 cells, MiaPaCa-2 cells treated for 24 h with 50, 100, 250, 500 and 1000 μmol/L of PEG-AuNPs nanoparticles complexed or not with NFL-BIOTIN peptide. Mitochondrial activity is expressed as mean % relative to the activity of control MiaPaCa-2 cells.

FIG. 5 shows the mitochondrial activity of MiaPaCa-2 cells, colchicine-treated MiaPaCa-2 cells and MiaPaCa-2 cells treated for 72 h with 50, 100, 250, 500 and 1000 μmol/L of PEG-AuNPs nanoparticles complexed or not with NFL-BIOTIN peptide. Mitochondrial activity is expressed as mean % relative to the activity of control MiaPaCa-2 cells.

FIG. 6 shows the mitochondrial activity of F98 cells, colchicine-treated F98 cells, F98 cells treated for 24 h with 50, 100, 250, 500 and 1000 μmol/L of PEG-AuNPs nanoparticles complexed or not with the NFL-BIOTIN peptide. Mitochondrial activity is expressed as mean % relative to the activity of control F98 cells.

FIG. 7 shows the mitochondrial activity of F98 cells, colchicine-treated F98 cells and F98 cells treated for 72 h with 50, 100, 250, 500 and 1000 μmol/L of PEG-AuNPs nanoparticles complexed or not with the NFL-BIOTIN peptide. Mitochondrial activity is expressed as mean % relative to the activity of control F98 cells.

FIG. 8 represents the cell viability of MiaPaCa-2 cells, treated with the peptide alone (NFL-BIOT) at 0.1 μM, with the PEG-AuNP nanoparticle (AuNPs) at 100 μM and with the NFL-BIOTINE-Au-NPs nanoparticle at 100 μM, after irradiation and internalization of the nanoparticles for 24 h. Cell viability is expressed as % relative to cell viability of untreated cells.

FIG. 9 represents the cell viability of MiaPaCa-2 cells, treated with peptide alone (NFL-BIOT) at 0.1 μM, with PEG-AuNP nanoparticle (AuNPs) at 100 μM and with NFL-BIOTINE-Au-NPs nanoparticle at 100 μM, after irradiation and internalization of the nanoparticles for 48 h. Cell viability is expressed as % relative to cell viability of untreated cells.

FIG. 10 shows the cell viability of F98 cells treated with the peptide alone (NFL-BIOT) at 0.1 μM, with the PEG-AuNP nanoparticle (AuNPs) at 100 μM and with the NFL-BIOTINE-Au-NPs nanoparticle at 100 μM, after irradiation and internalization of the nanoparticles for 24 h. Cell viability is expressed as % relative to cell viability of untreated cells.

FIG. 11 shows the cell viability of F98 cells treated with the peptide alone (NFL-BIOT) at 0.1 μM, with the PEG-AuNP nanoparticle (AuNPs) at 100 μM and with the NFL-BIOTINE-Au-NPs nanoparticle at 100 μM, after irradiation and internalization of the nanoparticles for 48 h. Cell viability is expressed as % relative to cell viability of untreated cells.

FIG. 12 shows the in vitro mitochondrial activity of rat glioblastoma cells (F98 cells) as a control, of F98 cells treated with colchicine (Col, 1 μg/mL) as a positive control and of F98 cells treated for 24 h with PEG-AuNPs, BIOT-TAT-PEGAuNPs and BIOT-VIM-PEG-AuNPs nanoparticles at different concentrations (0, 50, 100, 250, 500 or 1000 μmol/L). Mitochondrial activity was assessed by the MTS assay. Experiments were performed at least in triplicates. The symbol # means no cells present at the end of the incubation treatment. Data represent the mean ± standard error relative to the activity of control F98 cells. Statistical analysis was performed with Student's t-test (*p<0.05 and ***p<0.001).

FIG. 13 shows the in vitro mitochondrial activity of rat glioblastoma cells (F98) as a control, of F98 cells treated with colchicine (Col, 1 μg/mL) as a positive control and of F98 cells treated for 72 h with PEG-AuNPs, BIOT-TAT-PEGAuNPs and BIOT-VIM-PEG-AuNPs nanoparticles at different concentrations (0, 50, 100, 250, 500 or 1000 μmol/L). Mitochondrial activity was assessed by the MTS assay. Experiments were performed at least in triplicates. The symbol #means no cells present at the end of the incubation treatment. Data represent the mean ± standard error relative to the activity of control F98 cells. Statistical analysis was performed with Student's t-test (*p<0.05 and ***p<0.001).

FIG. 14 shows a transmission electron microscopy image illustrating the internalization of gold nanoparticles (PEG-AuNPs) in rat glioblastoma cells (F98). F98 cells were treated with nanoparticles at 250 μmol/L for 24 hours. N for nucleus and V for vacuoles. Scale bar (left): 2 μm. Scale bar (right): 50 nm.

FIG. 15 shows a transmission electron microscopy image illustrating the internalization of gold nanoparticles (PEG-AuNPs) coupled with the peptide TAT-BIOTIN (BIOT-TAT-PEGAuNPs) in rat glioblastoma cells (F98). F98 cells were treated with nanoparticles at 250 μmol/L for 24 h. N for nucleus and V for vacuoles. Scale bar (left): 2 μm. Scale bar (right): 50 nm.

FIG. 16 shows a transmission electron microscopy image illustrating the internalization of gold nanoparticles (PEG-AuNPs) coupled with the peptide VIM-BIOTIN (BIOT-VIM-PEGAuNPs) in rat glioblastoma cells (F98). F98 cells were treated with nanoparticles at 250 μmol/L for 24 h. N for nucleus and V for vacuoles. Scale bar (left): 2 μm. Scale bar (right): 50 nm.

FIG. 17 shows the average number of vacuoles containing more than 20 gold nanoparticles in F98 cells incubated for 24 h with 250 μmol/L PEG-AUNPs nanoparticles (black bar), BIOT-TAT-PEG-AUNPs nanoparticles (dark gray bar), and BIOT-VIM-PEG-AuNPs nanoparticles (light gray bar). Nanoparticles are counted manually on electron microscopy images.

### EXAMPLE 1

The inventors have synthesized and characterized NFL-BIOTIN-PEG-AuNPs gold nanoparticles according to the invention according to the process below.

### A. MATERIALS AND METHODS

### 1. Preparation of NFL-Biotin-PEG-AuNP Nanoparticles

Gold nanoparticles are mainly prepared by reduction of chloroauric acid (HAuCl₄, Sigma-Aldrich) at a concentration of 1 mmol/L.

After dissolving the gold salt, the solution is shaken vigorously, then 0.08 mL of biotinylated NFL peptide (NFL-BIOTIN) at a concentration of 1 mg/mL is added. The NFL peptide was previously diluted in 10% ethanol. Complexation of the peptide with HAuCl₄ gold salts is achieved by chelation.

Then, 250 μL of a stabilizing agent, PEG-COOH 600 (Poly-Ethylene-Glycol dicarboxylic, Sigma-Aldrich) was added at room temperature.

Finally, 1.2 mL at 3 mg/mL of reducing agent NaBH₄ (Sodium tetrahydruroborate, Sigma-Aldrich) is added, reducing Au³⁺ ions to neutral gold atoms (Au⁰).

The formation of NFL-BIOTIN-PEG-AuNPs nanoparticles is observed by the change in color of the solution from pale yellow to bright pink violet after the addition of the reducing agent.

The products of each synthesis stage are stored at 27-29° C. and characterized by UV-visible spectroscopy, Raman spectroscopy and transmission electron microscopy (TEM).

The NFL-BIOTIN-PEG-AuNPs solution was centrifuged at 10,000 rpm 3 times for 10 min, then the supernatant was discarded. The pellet was redispersed in an equivalent quantity of water. This was repeated twice to remove excess dicarboxylic PEG unconjugated to the NFL-BIOTIN peptide.

### 2. Nanoparticle Characterization

### 2.1. UV-Visible Absorption Spectroscopy

Absorption spectroscopy measurements were carried out using a UV-visible spectrophotometer (Uvikon 941 from Kontron instruments) controlled by Thermalys Uvikon 900 software. Absorption spectra were recorded in the spectral range between 200 and 900 nm. Solutions were placed in 1 cm quartz cuvettes.

### 2.2. Raman Spectroscopy

Raman spectra were recorded using an Xplora Raman microspectrometer (Horiba Scientifics) and processed using Labspec software. The spectrometer was set up with a HeNe laser (Helium-Neon) at 660 nm, a CCD camera for data acquisition and an optical filter set at 100% for a laser power of 8 mW and a network of 600 lines.

### 2.3 Zeta Potential Measurements

The zeta potential of NFL-BIOTIN-PEG-AuNPs dispersed in water was measured using the electrophoretic mode of a Zetasizer NanoZS (Malvern Instruments Ltd, Malvern, UK).

### 2.4. Dynamic Light Scattering (DLS)

The mean hydrodynamic diameters of NFL-BIOTIN-PEG-AuNPs nanoparticles dispersed in water were characterized using nanoparticle tracking analysis. Developed by NanoSight (Malvern Instruments Ltd), this equipment uses the properties of light scattering and Brownian motion to obtain particle size distributions of samples in liquid suspension. The measurement was carried out using an NS500 system equipped with a 405 nm laser, with version 3.0. Six sixty-second videos were recorded at a nanoparticle concentration sufficient to obtain a minimum of 200 terminated tracks per video for statistical significance. Data were recorded in ± standard deviation mode.

### 2.5. Transmission Electron Microscopy

Gold nanoparticles complexed or not with the NFL-BIOTIN peptide were observed by transmission electron microscopy. To this end, 2 μL of samples were deposited on 150-mesh copper grids, themselves coated with Formvar film for 1 min. The grids were then contrasted with 2% uranyl acetate for 1 min. Samples were observed using a Jeol microscope (model JEM-1400 with an accelerating voltage of 120 kV; Japan) equipped with a camera model 832 Orius SC-1000 of the brand Gatan.

### B. RESULTS

### 1. UV-Visible Characterization

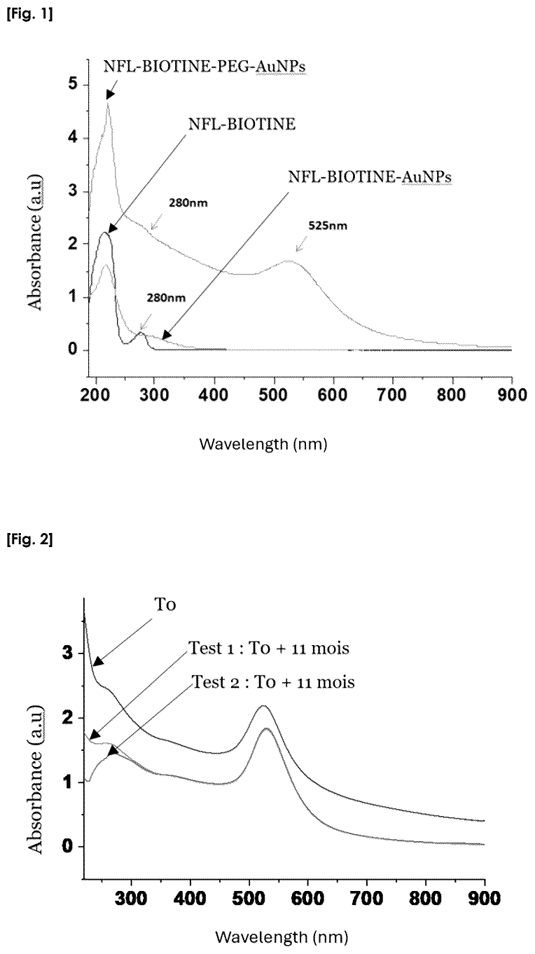

Complexation of HAuCl₄ with the NFL-BIOTIN peptide was observed, with a band at 300 nm representing HAuCl₄ and a band at 280 nm representing the NFL-BIOTIN peptide (see FIG. 1). A UV-visible band at 525 nm representing the plasmon band of NFL-BIOTIN-PEG-AuNPs nanoparticles was also observed. These results confirm the formation of NFL-BIOTIN-PEG-AuNPs nanoparticles. The 280 nm band, representing peptide control, is present at all stages of synthesis, confirming that the peptide is correctly grafted.

The results of DLS and zeta potential show that NFL-BIOTIN-PEG-AuNPs nanoparticles have a diameter of around 91±2 nm and a zeta potential of −24±1 mV.

### 2. Characterization by RAMAN Spectroscopy

Successful peptide grafting was also demonstrated by Raman spectroscopy. Raman spectra of the free NFL-BIOTIN peptide (control) show bands attributed to the amino acids that make up the peptide (tyrosine). A band 1449 cm⁻¹ represents the CH₂ group of biotin and a band 1656 cm⁻¹ is attributed to amide I. A broad band observed around 1600 cm⁻¹ is attributed to water. It was observed that with the formation of NFL-BIOTIN-PEG-AuNPs nanoparticles, we have a clearer fingerprint of the peptide signature, compared to the complex. As evidenced by these results, the formation of NFL-BIOTIN-PEG-AuNPs nanoparticles is confirmed, since nanoparticle formation exalts the signal.

### 3. Observing Nanoparticles With a Transmission Electron Microscope

Pegylated gold nanoparticles (PEG-AuNPs) complexed or not with the NFL-BIOTIN peptide were observed by transmission electron microscopy. These observations revealed a difference in nanoparticle shape in the absence or presence of the NFL-BIOTIN peptide. PEG-AuNPs nanoparticles are round in shape and relatively homogeneous in size.

When these same particles are complexed with the NFL-BIOTIN peptide, different particle shapes (round, rod, hexagon) are observed with fairly heterogeneous sizes.

### 4. Stability

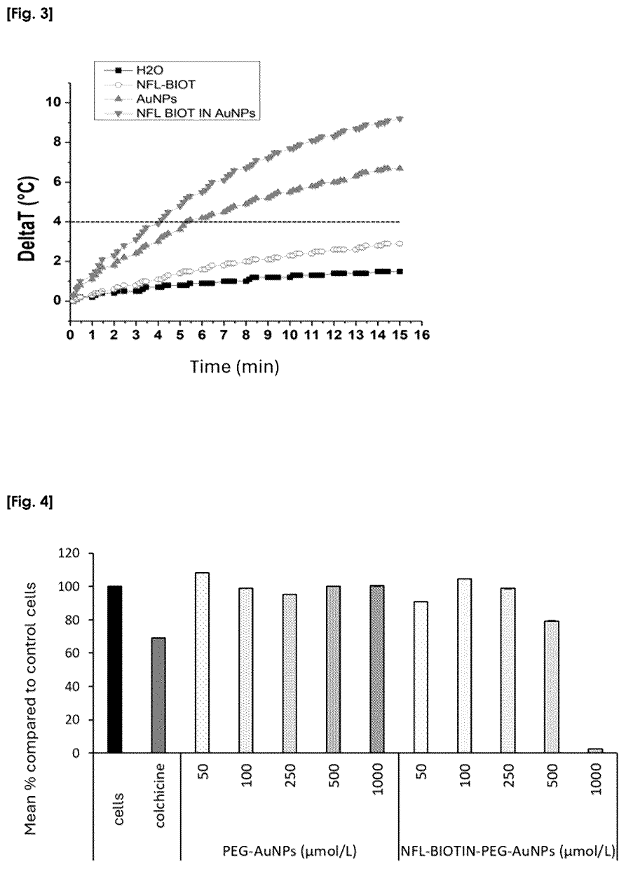

The bright red-violet color of the nanoparticles and the UV-visible spectra remain unchanged after storage at room temperature for 11 months, confirming that the formation of the nanoparticle suspension remains stable (see FIG. 2).

### EXAMPLE 2

The inventors evaluated the effect of NFL-Biotin-PEG-AuNP gold nanoparticles on two cancer cell lines.

### A. MATERIALS AND METHODS

### 1. Cell Lines Used

Two cell lines obtained from American Tissue Culture Collection (ATCC) were used for this study:
MiaPaCa-2 cells, a human pancreatic cancer cell line, and
F98 cells, a rat glioblastoma cell line.

Both cell lines were cultured in DMEM medium (Dulbecco's Modified Eagle's medium; Gibco, Bio-Sciences Ltd, Ireland) supplemented with 10% FBS (Fetal Bovine Serum; Sigma-Aldrich), 1% antibiotics (penicillin at 50 IU/mL and streptomycin at 50 μg/mL) and L-glutamine (2 mmol/L).

### 2. Measurement of Cellular Mitochondrial Activity

To investigate the effects of gold nanoparticles complexed or not with the NFL-BIOTIN peptide on cell viability, MTS cell viability assays (ab197010; Abcam, Paris, France) measuring mitochondrial activity in cells were performed.

These tests were carried out on both cell types. Briefly, cells were seeded in 96-well plates at 1000 or 3000 cells per well (cell numbers varying according to treatment duration) and incubated for 24 hours at 37° C. and 5% CO₂. The various treatments were then applied to the cells: colchicine (1 μg/mL, C9754; Sigma-Aldrich) or concentrations of gold nanoparticles complexed or not with the NFL-BIOTIN peptide (between 50 and 1000 μmol/L) for 24 or 72 hours at 37° C. and 5% CO₂. At the end of the treatment (24 or 72 hours), 20 μL of MTS reagent was added to each well for 4 hours. Absorbance at 490 nm was measured using a Spectra Max M2 spectrophotometer (Molecular Devices, San Jose, California, USA).

### 3. Internalization of the Nanoparticle Observed by Transmission Electron Microscopy

Cells (MiaPaCa-2 and F98) were seeded in 6-well plates at 400,000 cells per well and incubated for 24 hours at 37° C. and 5% CO₂. Then, nanoparticles at 100 or 500 μmol/L without or with peptide at 0.08 mmol/L were incubated with the cells for 24 or 72 hours. After incubation, the cells were washed with 0.1 mol/L phosphate buffer (pH 7.4) and fixed overnight at 4° C. with a solution of 2.5% glutaraldehyde in 0.1 mol/L phosphate buffer (pH 7.4). Cells were then rinsed with 0.1 mol/L phosphate buffer. The cells were then rinsed with distilled water and post-fixed in a 1% osmium tetroxide solution for 1 hour. They were then rinsed 3×5 min with water, and incubated 15 min in 50° ethanol, 15 min in 70° ethanol, 15 min in 95° ethanol and 3×30 min in 100° ethanol. They were then placed in a mixture 50% ethanol 100°/50% Epon resin (volume/volume) and incubated overnight. The next day, the remaining diluted Epon resin was removed and replaced by a pure Epon bath for 4 hours, then this Epon bath was replaced by another pure Epon bath. The plate with the cells in the Epon was placed for 24 hours at 37° C., then 24 hours at 45° C. and finally 72 hours at 60° C. Once the resin had polymerized at 60° C., 60 nm-thick ultrathin sections were cut with a UC7 ultramicrotome (Leica, Wetzlar, Germany) and deposited on 150 Mesh copper grids. The sections were then contrasted with a solution of 3% uranyl acetate in 50° ethanol for 15 min, then rinsed with ultrapure water. Samples were then observed using a Jeol microscope (model JEM-1400 with 120 kV accelerating voltage; Japan) equipped with a camera model 832 Orius SC-1000 of the brand Gatan.

### 4. Photothermia on Cell Lines

MiaPaCa-2 and F98 cells were seeded at a density of 200,000 cells/mL in 25 cm² culture flasks and grown at 37° C. and 5% CO₂. Cells were then seeded in 96-well plates at 200 μL of cells per well, and left for 24 or 48 hours. Then, 50 μL of medium per well were removed and replaced by nanoparticle solutions, which were incubated for 24 hours. The medium was then removed, and the cells were washed three times with PBS (Phosphate Buffered Saline), to remove the excess non-internalized nanoparticles. The same volume of medium per well was then added. Each well of the plate was subjected to an 808 nm laser source with a power of 0.5 W/cm². Experiments were carried out beforehand to define the optimum parameters, to avoid any risk of artifact due to the laser parameter. Studies were carried out at two times: 5 and 10 min, to see if the “time” parameter could have an impact on the results. After subjecting the cells to radiation from the 808 nm near-infrared laser, the medium was changed and left for 24 hours, then a cell viability test was carried out to check that the nanoparticles had the same effect on the cells before and after irradiation treatment. To perform photothermy, 1 mL of the nanoparticle solution was deposited in a tank and the nanoparticles were heated using an infrared laser. A thermal probe was then used to record the temperature rise over a 15-minute period.

### B. RESULTS

### 1. Photothermalization of Nanoparticles Alone

FIG. 3 shows that the temperature rises above the 4° C. threshold. NFL-BIOTIN-PEG-AuNPs nanoparticles are therefore excellent candidates for photothermal testing.

### 2. Impact of NFL-BIOTIN-PEG-AuNPs on Cell Lines

### 2.1 Impact of Nanoparticles on Mitochondrial Activity

To assess mitochondrial activity, both cell lines were treated with 1 μg/mL colchicine or with 0, 50, 100, 250, 500 or 1000 μmol/L PEG-AuNPs nanoparticles complexed or not with the NFL-BIOTIN peptide for 24 or 72 hours. The action of colchicine is to interact with tubulin and disrupt microtubule assembly (Bhattacharyya et al., 2008), resulting in cell death. Colchicine therefore serves as a positive control.

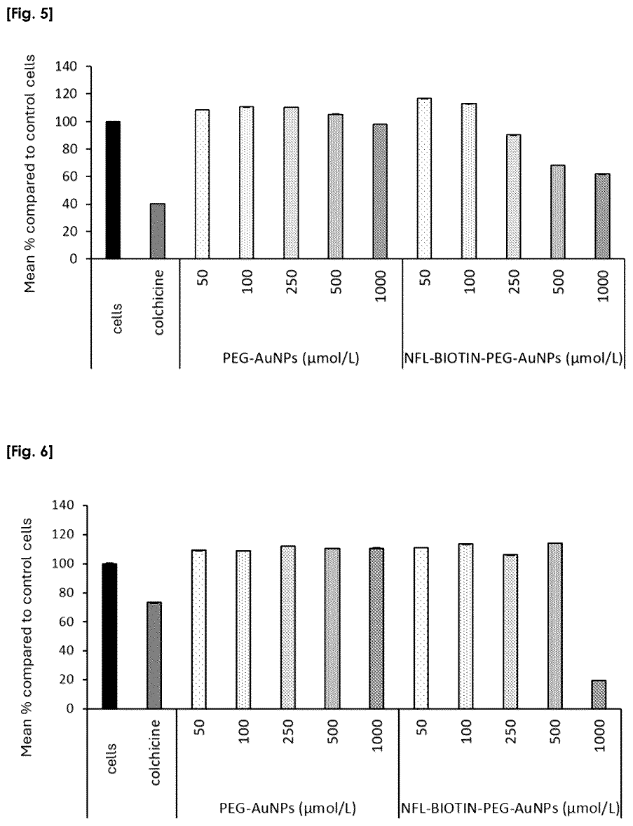

For MiaPaCa-2 cells treated for 24 h, the MTS assay showed no effect of PEG-AuNPs, however a decrease in mitochondrial activity was observed from a treatment with 500 μmol/L NFL-BIOTIN-PEG-AuNPs, and a very strong decrease was observed at the 1000 μmol/L dose (FIG. 4).

For MiaPaCa-2 cells treated for 72 hours, the MTS assay shows similar results, i.e. no effect of PEG-AuNPs, and a decrease in mitochondrial activity starting with treatment with 500 μmol/L NFL-BIOTIN-PEG-AuNPs (FIG. 5).

The same tests were carried out on F98 rat glioblastoma cells. Similar results were observed, with no effect of PEG-AuNPs on mitochondrial activity after 24 h treatment, and a drastic reduction in mitochondrial activity when the cells were treated with 1000 μmol/L NFL-BIOTIN-PEG-Au-NPs (FIG. 6).

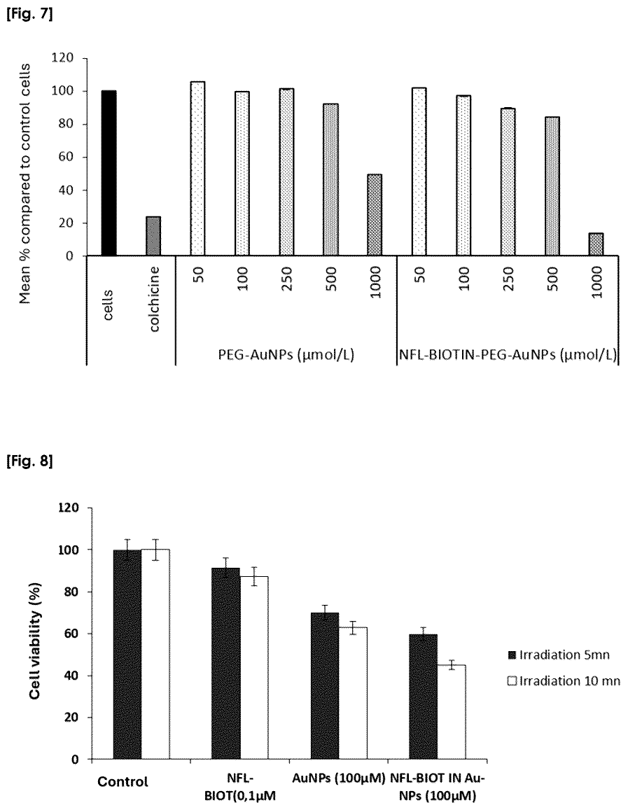

When F98 cells were treated for 72 h with nanoparticles, a reduction in mitochondrial activity with both particle types was observed at 1000 μmol/L (FIG. 7).

### 2.2 Cellular Internalization of Nanoparticles

MiaPaCa-2 and F98 cells were treated for 72 h with PEG-AuNPs or NFL-BIOTIN-PEG-AuNPs at 500 μmol/L. In MiaPaCa-2 cells, nanoparticles with or without peptides entered the cells, mainly in vacuoles. Some nanoparticles are trapped in spaces between cells. These observations also reveal the different particle shapes and the heterogeneity of particle sizes.

The same observations were made with F98 cells.

### 2.3. Photothermia on MiaPaCa-2 Pancreas and F98 Glioblastoma Cell Lines

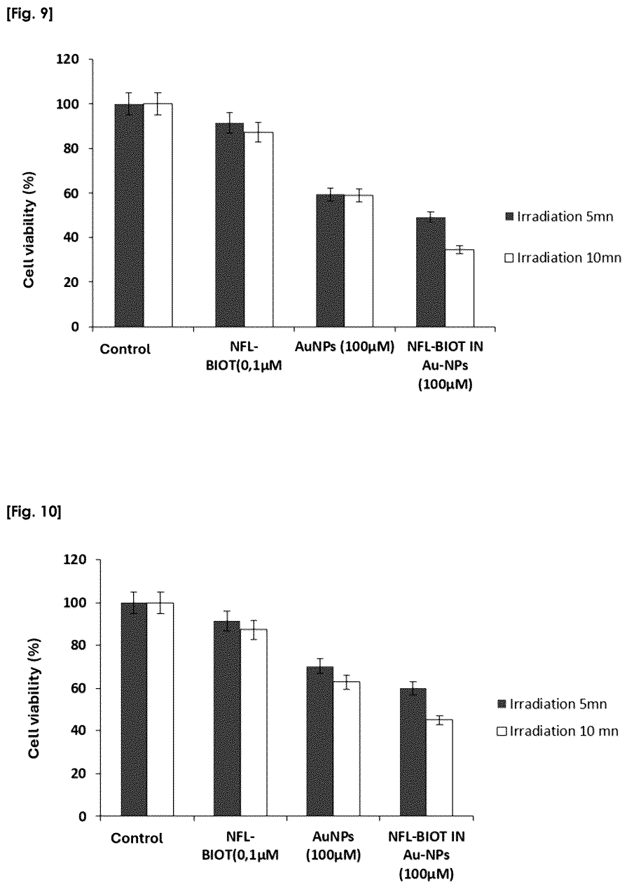

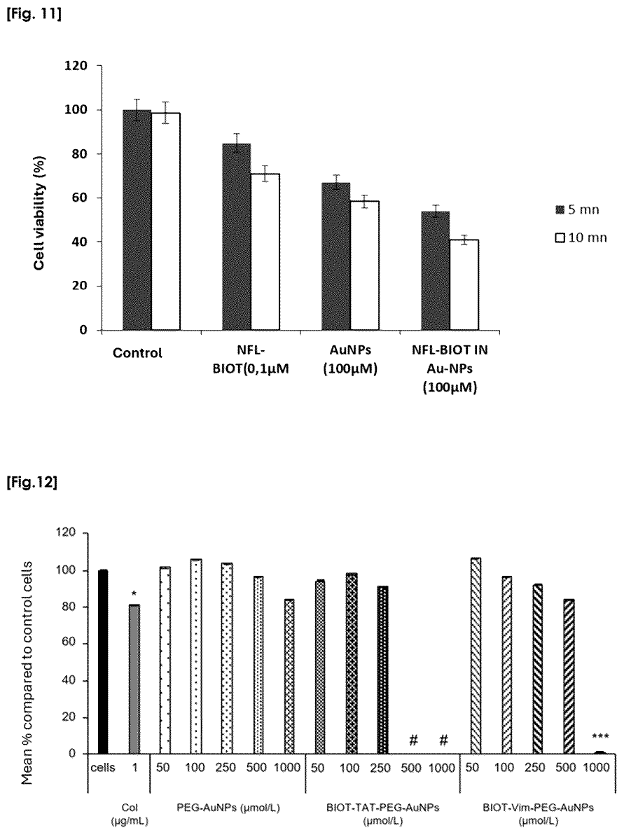

FIGS. 8 to 11 show that irradiation treatment had no effect on cells alone without nanoparticles (control, 100% cell viability). With the peptide, a similar effect was observed with or without irradiation, whatever the internalization time. For PEG-AuNPs nanoparticles without peptide, a decrease in cell viability was observed, with cell viability around 60% relative to the cell viability obtained with the control after 24 h and 48 h of internalization. For NFL-BIOTIN-PEG-AuNPs nanoparticles, a significant decrease in cell viability was observed, with cell viability around 60% for 5 min of irradiation and around 45% for 10 min of irradiation after 24 h of internalization. Furthermore, cell viability is around 40% for 5 min irradiation and around 30% for 10 min after 48 h of internalization.

### EXAMPLE 3

The inventors have synthesized gold nanoparticles TAT-BIOTIN-PEG-AuNPs and VIM-BIOTIN-PEG-AuNPs according to the invention according to a process summarized above similar to the process described in the previous Example 1. Gold nanoparticles are prepared by reducing chloroauric acid (HAuCl₄, Sigma-Aldrich) to a concentration of 1 mmol/L.

After dissolving the gold salt, biotinylated TAT peptide (TAT.48-60 peptide; TAT-BIOTIN-peptide; BIOT-GRKKRRQRRPPQ-CONH₂; Millegen, Toulouse, France) or biotinylated VIM peptide (VIM-BIOTIN-peptide; BIOT-GGAYVTRSSAVRLRSSVPGVRLLQ-CONH₂; Millegen, Toulouse, France) is added. The solution is stirred vigorously for 10 min. Complexation of the peptide with HAuCl₄ gold salts is achieved by chelation.

A stabilizing agent, PEG-COOH 600 (Poly-Ethylene-Glycol dicarboxylic, Sigma-Aldrich) is then added at room temperature.

Then, a reducing agent NaBH₄ (Sodium tetrahydruroborate, Sigma-Aldrich) is added, reducing Au³⁺ ions to neutral gold atoms (Au⁰).

The formation of TAT-BIOTIN-PEG-AuNPs and VIM-BIOTIN-PEG-AuNPs nanoparticles is observed through a color change of the solution from pale yellow to bright pink violet after the addition of the reducing agent.

### EXAMPLE 4

The inventors evaluated the effect of gold nanoparticles TAT-BIOTIN-PEG-AuNPs and VIM-BIOTIN-PEG-AuNPs on a cancer cell line.

### A. MATERIALS AND METHODS

### 1. Cell Line Used

For this study, the F98 cell line, a rat glioblastoma line, obtained from American Tissue Culture Collection (ATCC) was used.

The cell line was cultured in DMEM (Dulbecco's Modified Eagle's medium; Sigma-Aldrich) supplemented with 10% FBS (Fetal Bovine Serum; Sigma-Aldrich), 1% antibiotics (penicillin 50 IU/mL and streptomycin 50 μg/mL) and L-glutamine (2 mmol/L).

### 2. Measurement of Cellular Mitochondrial Activity

To investigate the effects of gold nanoparticles complexed or not with the TAT-BIOTIN or VIM-BIOTIN peptide on cell viability, MTS cell viability assays (ab197010; Abcam, Paris, France) measuring mitochondrial activity in cells were performed.

These tests were performed on F98 cells. Briefly, cells were seeded in 96-well plates at 1000 or 3000 cells per well (cell numbers varying according to treatment duration) and incubated for 24 hours at 37° C. and 5% CO₂. The cells were then treated with colchicine (1 μg/mL, C9754; Sigma-Aldrich) or concentrations of gold nanoparticles complexed or not with the VIM-BIOTIN or TAT-BIOTIN peptide (between 50 and 1000 μmol/L) for 24 or 72 hours at 37° C. and 5% CO₂. At the end of treatment (24 or 72 hours), 20 μL of MTS reagent was added to each well for 4 hours. Absorbance at 490 nm was measured using a Spectra Max M2 spectrophotometer (Molecular Devices, San Jose, California, USA).

### 3. Internalization of Nanoparticle Observed by Transmission Electron Microscopy

F98 cells were seeded in 12-well plates at 100,000 cells per well and incubated for 24 h at 37° C. and 5% CO₂. Then, PEG-AuNPs (at 500 μmol/L), VIM-BIOTIN-PEG-AuNPs (at 250 μmol/L) or TAT-BIOTIN-AuNPs (at 250 μmol/L) nanoparticles were incubated with the cells for 24 or 72 hours. After incubation, cells were washed with 0.1 mol/L phosphate buffer (pH 7.4) and fixed overnight at 4° C. with a solution of 2.5% glutaraldehyde in 0.1 mol/L phosphate buffer (pH 7.4). Cells were then rinsed with 0.1 mol/L phosphate buffer. The cells were then rinsed with distilled water and post-fixed in a 1% osmium tetroxide solution for 1 hour. They were then rinsed 3×5 min with water, and incubated 15 min in 50° ethanol, 15 min in 70° ethanol, 15 min in 95° ethanol and 3×30 min in 100° ethanol. They were then placed in a mixture 50% ethanol 100°/50% Epon resin (volume/volume) and incubated overnight. The next day, the remaining diluted Epon resin was removed and replaced by a pure Epon bath for 4 hours, then this Epon bath was replaced by another pure Epon bath. The plate with the cells in the Epon was placed for 24 hours at 37° C., then 24 hours at 45° C. and finally 72 hours at 60° C. Once the resin had polymerized at 60° C., 60 nm-thick ultrathin sections were cut with a UC7 ultramicrotome (Leica, Wetzlar, Germany) and deposited on 150 Mesh copper grids. The sections were then contrasted with a solution of 3% uranyl acetate in 50° ethanol for 15 min, then rinsed with ultrapure water. Samples were then observed using a Jeol microscope (model JEM-1400 with 120 kV accelerating voltage; Japan) equipped with a camera model 832 Orius SC-1000 of the brand Gatan.

### B. RESULTS

### 1. Impact of Nanoparticles on Mitochondrial Activity

To assess mitochondrial activity, F98 cells were treated with 1 μg/mL colchicine or with 0, 50, 100, 250, 500 or 1000 μmol/L PEG-AuNPs nanoparticles complexed or not with VIM-BIOTIN peptide or TAT-BIOTIN peptide for 24 or 72 hours. Similar to the experiments described in Example 2 above, colchicine was used as a positive control.

For F98 cells treated for 24 h, the MTS assay showed no effect of PEG-AuNPs on mitochondrial activity. On the other hand, a significant decrease in mitochondrial activity was observed from a treatment with 1000 μmol/L VIM-BIOTIN-PEG-AuNPs, and a very strong toxicity was observed for treatments with 500 μmol/L and 1000 μmol/L TAT-BIOTIN-PEG-AuNPs. Indeed, for these two treatment concentrations with TAT-BIOTIN-PEG-AuNPs nanoparticles, not a single cell was viable after 24 hours (FIG. 12).

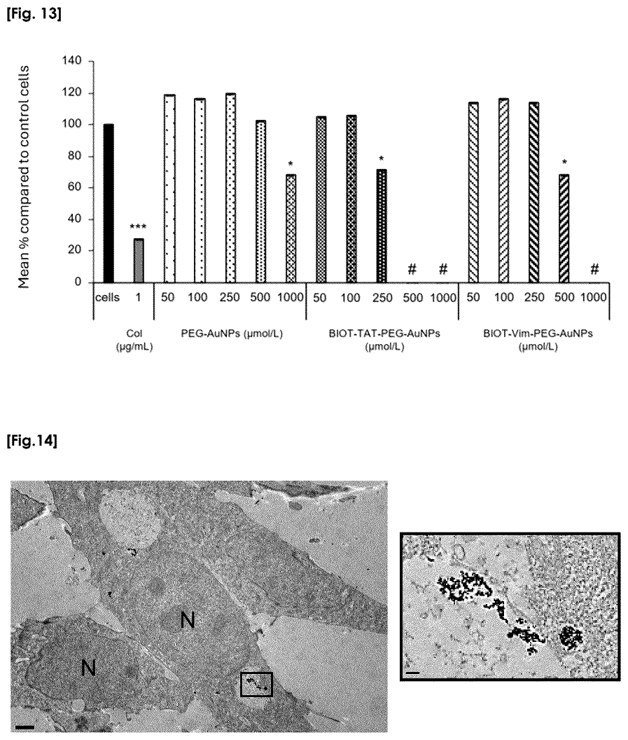

For F98 cells treated for 72 hours, the MTS assay shows similar results at lower concentrations. In fact, a significant decrease in mitochondrial activity was observed from treatment with 1000 μmol/L PEG-AuNPs, 250 μmol/L TAT-BIOTIN-PEG-AuNPs or 500 μmol/L VIM-BIOTIN-PEG-AuNPs. In addition, the strong toxicity induced is found from 500 μmol/L of TAT-BIOTIN-PEG-AuNPs and also manifests itself from 1000 μmol/L of VIM-BIOTIN-PEG-AuNPs (FIG. 13).

Neither the function nor the biological activity of VIM-BIOTIN and TAT-BIOTIN peptides was affected by the combination with PEG-AuNPs nanoparticles.

### 2. Cellular Internalization of Nanoparticles

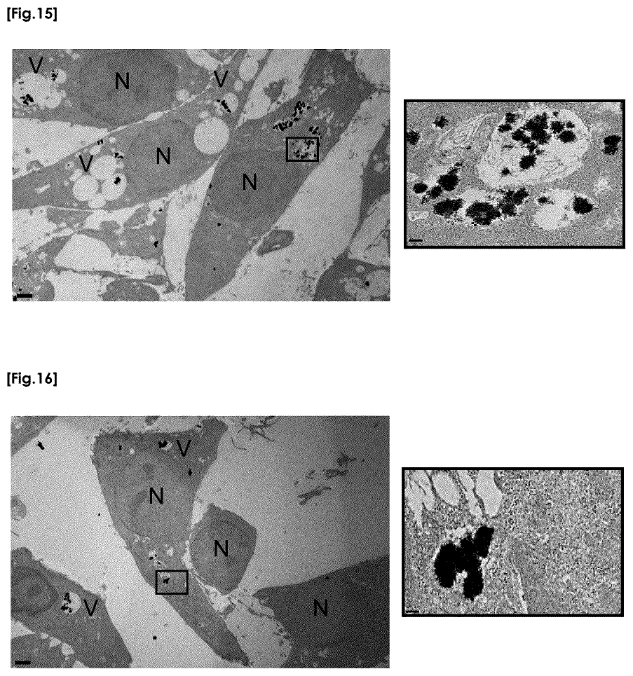

F98 cells were treated for 24 h with VIM-BIOTIN-PEG-AuNPs or TAT-BIOTIN-PEG-AuNPs nanoparticles at 250 μmol/L. Transmission electron microscopy images show F98 cells treated with PEG-AuNPs nanoparticles (FIG. 14), VIM-BIOTIN-PEG-AuNPs (FIG. 15) or TAT-BIOTIN-PEG-AuNPs (FIG. 16).

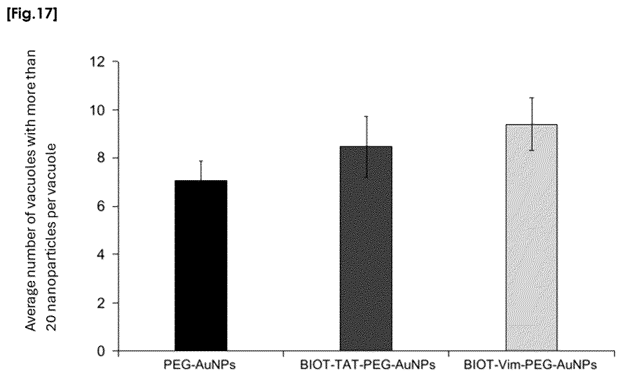

For cells treated with VIM-BIOTIN-PEG-AuNPs or TAT-BIOTIN-PEG-AuNPs nanoparticles, more vacuoles were detected. The nanoparticles were quantified by manual counting (FIG. 17). It can be seen that the average number of vacuoles containing more than 20 nanoparticles is higher for VIM-BIOTIN-PEG-AuNPs and TAT-BIOTIN-PEG-AuNPs nanoparticles than for PEG-AuNPs nanoparticles.

1. A nanoparticle comprising:
an association of at least one metal salt and at least one peptide; and
at least one biocompatible polymer.

2. The nanoparticle according to claim 1, wherein the metal salt is chelated by the peptide.

3. The nanoparticle according to claim 1, wherein the metal salt and the peptide are encapsulated by the biocompatible polymer.

4. The nanoparticle according to claim 1, wherein the nanoparticle has been reduced by a reducing agent.

5. The nanoparticle according to claim 1, wherein the metal salt is selected from the group consisting of a salt of gold (Au), selenium (Se), gadolinium (Gd), cobalt (Co), europium (Eu), terbium (Tb), cerium (Ce), manganese (Mn), iron (Fe), zinc (Zn) and copper (Cu).

6. The nanoparticle according to claim 1, wherein the metal salt is chloroauric acid HAuCl₄.

7. The nanoparticle according to claim 1, wherein the peptide comprises from 2 to 40 amino acids.

8. The nanoparticle according to claim 1, wherein the peptide is the NFL peptide having the following amino acid sequence: YSSYSAPVSSSLSVRRSYSSSSGS, the TAT peptide having the following amino acid sequence: GRKKRRQRRRPPQ, the VIM peptide having the following amino acid sequence: GGAYVTRSSAVRLRSSVPGVRLLQ, the transportane peptide having the following amino acid sequence: GWTLNSAGYLLGKINLKALAALAKKIL, the penetratin peptide having the following amino acid sequence: RQIKIWFQNRRMKWKK, the MAP peptide having the following amino acid sequence: KLALKLALKALKAALKLA, the Pep-1 peptide having the following amino acid sequence: KETWWETWWTEWSQPKKKRKV, the Pept 1 peptide having the following amino acid sequence: PLILLRLLRGQF, the Pept 2 peptide having the following amino acid sequence: PLIYLRLLRGQF, the IVV-14 peptide having the following amino acid sequence: KLWMRWYSPTTRRYG, the Ig(v) peptide having the following amino acid sequence: MGLGLHLLVLAAALQGAKKKRKV, the pVEC peptide having the following amino acid sequence: LLIILRRRIRKQAHAHSK, the ppTG20 peptide having the following amino acid sequence: GLFRALLRLLRSLWRLLLRA, the APP peptide having the following amino acid sequence: APP GLARALTRLLRQLTRQLTRA, the peptide having the following amino acid sequence RLWMRWYSPRTRAYGC; the peptide having the following amino acid sequence: WRWYCR, the PepFect 14 peptide having the following amino acid sequence: stearyl-AGYLLGKLLOOLAAAALOOLL-NH2, the peptide having the following amino acid sequence: GWTLNSAGYLLGKINLKALAALAKKIL, the HRSV peptide having the following amino acid sequence: RRIPNRRPRR, or a derivative thereof.

9. The nanoparticle according to claim 1, wherein the peptide is selected from the NFL peptide having the following amino acid sequence: YSSYSAPVSSSLSVRRSYSSSSGS, the TAT peptide having the following amino acid sequence: GRKKRRQRRRPPQ or the VIM peptide with the following amino acid sequence: GGAYVTRSSAVRLRSSVPGVRLLQ.

10. The nanoparticle according to claim 1, wherein the biocompatible polymer is a thermosensitive polymer.

11. The nanoparticle according to claim 1, wherein the biocompatible polymer is selected from the group consisting of polyethylene glycol, polyethylene glycol diacid, polyethylene glycol diamine, collagen, alginate, chitosan, dextran, poly(N-isopropylacrylamide), poly[2-(dimethylamino)ethyl methyl methacrylate] (pDMAEMA), hydroxypropyl cellulose, poly(vinylcaprolactam), polyvinyl methyl ether and combinations thereof.

12. The nanoparticle according to claim 1, wherein the biocompatible polymer is dicarboxylic polyethylene glycol.

13. The nanoparticle according to claim 1, wherein the nanoparticle has a diameter between 20 and 200 nm.

14. A drug and/or diagnostic agent comprising the nanoparticle according to claim 1.

15. A method for the prevention or treatment of glioblastoma or pancreatic cancer in an individual, comprising administering an effective amount of at least one nanoparticle according to claim 1 to the individual.

16. A pharmaceutical composition comprising at least one nanoparticle according to claim 1 as active ingredient, optionally in association with a pharmaceutically acceptable carrier.

17. A diagnostic composition comprising at least one nanoparticle according to claim 1.

18. A medical device comprising at least one nanoparticle according to claim 1.

19. A method of preparing a nanoparticle according to claim 1, comprising:
chelating a metal salt with a peptide to obtain a chelated metal salt;
mixing the chelated metal salt with a biocompatible polymer to obtain a mixture; and
reducing the mixture with a reducing agent.

20. The process according to claim 19, wherein the process is carried out in the absence of a chemical surfactant other than the biocompatible polymer and in the absence of a chemical binder.


SEQUENCE LISTING (19 sequences)

SEQ ID NO: 1 - protein, 24 aa, synthetic construct
  REGION 1..24: note = PEPTIDE NFL
YSSYSAPVSSSLSVRRSYSSSSGS

SEQ ID NO: 2 - protein, 13 aa, synthetic construct
  REGION 1..13: note = PEPTIDE TAT
GRKKRRQRRRPPQ

SEQ ID NO: 3 - protein, 24 aa, synthetic construct
  REGION 1..24: note = PEPTIDE VIM
GGAYVTRSSAVRLRSSVPGVRLLQ

SEQ ID NO: 4 - protein, 27 aa, synthetic construct
  REGION 1..27: note = PEPTIDE TRANSPORTANE
GWTLNSAGYLLGKINLKALAALAKKIL

SEQ ID NO: 5 - protein, 16 aa, synthetic construct
  REGION 1..16: note = PEPTIDE PENETRATINE
RQIKIWFQNRRMKWKK

SEQ ID NO: 6 - protein, 18 aa, synthetic construct
  REGION 1..18: note = PEPTIDE MAP
KLALKLALKALKAALKLA

SEQ ID NO: 7 - protein, 21 aa, synthetic construct
  REGION 1..21: note = PEPTIDE PEP-1
KETWWETWWTEWSQPKKKRKV

SEQ ID NO: 8 - protein, 12 aa, synthetic construct
  REGION 1..12: note = PEPTIDE PEPT 1
PLILLRLLRGQF

SEQ ID NO: 9 - protein, 12 aa, synthetic construct
  REGION 1..12: note = PEPTIDE PEPT 2
PLIYLRLLRGQF

SEQ ID NO: 10 - protein, 15 aa, synthetic construct
  REGION 1..15: note = PEPTIDE IVV-14
KLWMRWYSPTTRRYG

SEQ ID NO: 11 - protein, 23 aa, synthetic construct
  REGION 1..23: note = PEPTIDE IG(V)
MGLGLHLLVLAAALQGAKKKRKV

SEQ ID NO: 12 - protein, 18 aa, synthetic construct
  REGION 1..18: note = PEPTIDE PVEC
LLIILRRRIRKQAHAHSK

SEQ ID NO: 13 - protein, 20 aa, synthetic construct
  REGION 1..20: note = PEPTIDE PPTG20
GLFRALLRLLRSLWRLLLRA

SEQ ID NO: 14 - protein, 20 aa, synthetic construct
  REGION 1..20: note = PEPTIDE APP
GLARALTRLLRQLTRQLTRA

SEQ ID NO: 15 - protein, 16 aa, synthetic construct
  REGION 1..16: note = PEPTIDE DE PENETRATION CELLULAIRE
RLWMRWYSPRTRAYGC

SEQ ID NO: 16 - protein, 6 aa, synthetic construct
  REGION 1..6: note = PEPTIDE DE PENETRATION CELLULAIRE
WRWYCR

SEQ ID NO: 17 - protein, 21 aa, synthetic construct
  REGION 1..21: note = PEPTIDE DE PENETRATION CELLULAIRE
  SITE 10: note = MISC_FEATURE - Ornithine
  SITE 11: note = MISC_FEATURE - Ornithine
  SITE 18: note = MISC_FEATURE - Ornithine
  SITE 19: note = MISC_FEATURE - Ornithine
AGYLLGKLLXXLAAAALXXLL

SEQ ID NO: 18 - protein, 27 aa, synthetic construct
  REGION 1..27: note = PEPTIDE DE PENETRATION CELLULAIRE
GWTLNSAGYLLGKINLKALAALAKKIL

SEQ ID NO: 19 - protein, 10 aa, synthetic construct
  REGION 1..10: note = PEPTIDE HRSV
RRIPNRRPRR